(19) United States
(12) Patent Application Publication
(10) Pub. No.: US 2011/0131841 A1
(43) Pub. Date: Jun. 9, 2011

(54) ARTICLE OF FOOTWEAR

(76) Inventor: DAVID W. FOSTER, Bolton (GB)

(21) Appl. No.: 12/879,181

(22) Filed: Sep. 10, 2010

Related U.S. Application Data
(63) Continuation of application No. PCT/GB2009/000631, filed on Mar. 9, 2009.

(30) Foreign Application Priority Data
Mar. 13, 2008 (GB) 0804643.5

(51) Int. Cl. A43B 13/14; A43B 7/14; A61F 5/14

(52) U.S. Cl. 36/25 R; 36/142; 36/143; 36/144

(57) ABSTRACT

The present invention relates to a control element (3) for installation in a sole (5) of an article of footwear (1). The control element (3) comprises a rearfoot portion (13), a central portion (15) and a forefoot portion (17). The forefoot portion (17) is suitable for supporting medial and/or lateral portions of a wearer's foot. The central portion (15) joins the rearfoot portion (13) and the forefoot portion (17). The rearfoot portion (13) provides an inclined support (19) for controlling pronation. The present invention also relates to an article of footwear (1) incorporating a control element (3) therein.

### RELATED APPLICATIONS

This application is a continuation of PCT/GB2009/000631, filed on Mar. 9, 2009 which claims priority to GB patent application no. 0804643.5 filed on Mar. 13, 2008. The contents of each of these applications is incorporated herein by reference in their entirety.

The present invention relates to a control element for installation in the sole of an article of footwear. The invention also relates to an article of footwear having a control element installed in the sole.

It is known from U.S. Pat. No. 6,199,303 to provide a stability element for controlling the rotation of the forefoot portion of an article of footwear. The stability element is intended to control either pronation or supination and may extend substantially along the medial side of the shoe or substantially along the lateral side. The particular bending properties of the stability element serve to control pronation or supination as appropriate.

Furthermore, a torsional spring insole for use in footwear is known from U.S. Pat. No. 5,179,791. An elevated heel section is connected to an inner sole via a step-down region. The inner sole flexes in response to a pressure imbalance applied to the elevated heel section to eliminate the pressure imbalance and to counteract pronation and supination in the foot.

The known systems rely on the flexural properties of the sole to control pronation or supination of the foot. Accordingly, they may not offer sufficient control or correction during the initial phase of a stepping motion when the sole is not loaded. Moreover, the flexing of the sole may vary depending on the loads applied and, therefore, may offer varying levels of control depending on the activity being performed.

Alternative reinforcing elements for the soles of articles of footwear are known from U.S. Pat. No. 6,954,998 and U.S. Pat. No. 7,082,702. However, there is no suggestion that these elements may control supination or pronation.

The present invention, at least in preferred embodiments, attempts to ameliorate or overcome at least some of the problems with known systems.

Viewed from a first aspect, the present invention relates to a control element for installation in a sole of an article of footwear, the control element comprising a rearfoot portion; a forefoot portion for supporting medial and/or lateral portions of a wearer's foot; and a central portion joining the rearfoot portion and the forefoot portion; wherein said rearfoot portion provides a first inclined support for controlling pronation. The rearfoot portion of the control element is preferably suitable for supporting a medial side of a wearer's foot above a lateral side thereof. Thus, at least in preferred embodiments, when the control element is installed in the article of footwear, pronation may be controlled.

The first inclined support is preferably inclined in a transverse direction. Preferably, the first inclined support operatively slopes downwardly from a medial side of the control element towards a lateral side thereof. The rearfoot portion preferably comprises a first raised section that forms said first inclined support. The first raised section preferably forms an inclined support surface, which may be real or virtual.

The first inclined support is preferably operatively inclined at an angle between 2° and 10° relative to the ground. More preferably, the first inclined support is operatively inclined at an angle between 4° and 8° relative to the ground. At least in preferred embodiments, the first inclined support may compensate for pronation in the ankle. The angle of variance of the knee to the hip joint, which is reflected in the degree of pronation in the ankle, is less for men than for women so the control element may be tailored depending on whether the article of footwear is intended to be worn by a man or a woman.

In preferred arrangements, the forefoot portion may form a second inclined support for controlling supination. The forefoot portion of the control element is preferably suitable for supporting a lateral side of a wearer's foot above a medial side thereof. Thus, at least in preferred embodiments, when the control element is installed in the article of footwear, supination may also be controlled.

The second inclined support is preferably inclined in a transverse direction. Preferably, the second inclined support operatively slopes downwardly from a lateral side of the control element towards a medial side thereof. The forefoot portion preferably comprises a second raised section forming said second inclined support.

The second inclined support is preferably operatively inclined at an angle between 1° and 8° relative to the ground. More preferably, the second inclined support is operatively inclined at an angle between 2° and 4° relative to the ground.

The first and second raised sections may be separate from each other. Preferably, however, the first and second raised sections are joined to form a continuous raised section. The continuous raised section may, for example, extend from the rearfoot portion to the forefoot portion of the control element. The continuous raised section preferably extends diagonally across the central portion of the control element. Preferably, the continuous raised section extends from a medial side of the rearfoot portion to a lateral side of the forefoot portion.

The first raised section and/or the second raised section may have a honeycomb structure. This may help reduce the weight of the control element. The honeycomb structure preferably comprises a plurality of cells. The cells are preferably operatively arranged substantially vertically.

Rather than provide a first raised section and/or a second raised section, the forefoot portion may be twisted relative to the rearfoot portion about a longitudinal axis of the control element. The angular offset of the forefoot portion relative to the rearfoot portion may provide the desired control of the supination and/or pronation. Alternatively, the rearfoot portion and/or the forefoot portion may be tapered to provide the first inclined support and/or the second inclined support. For example, the rearfoot portion and/or the forefoot portion may be tapered in a transverse direction to control supination and/or pronation.

Viewed from a further aspect, the present invention relates to a control element for installation in a sole of an article of footwear, the control element comprising a rearfoot portion; a forefoot portion for supporting medial and/or lateral portions of a wearer's foot; and a central portion joining the rearfoot portion and the forefoot portion; wherein said forefoot portion forms an inclined support for controlling supination. The forefoot portion of the control element is preferably suitable for supporting a lateral side of a wearer's foot above a medial side thereof. Thus, at least in preferred embodiments, when the control element is installed in the article of footwear, supination may be controlled. The inclined support is preferably operatively inclined downwardly from a lateral side of the control element towards a medial side thereof.

Viewed from a still further aspect, the present invention relates to a control element for installation in a sole of an article of footwear, the control element comprising a rearfoot portion; a forefoot portion for supporting medial and/or lateral portions of a wearer's foot; and a central portion joining the rearfoot portion and the forefoot portion; wherein the rearfoot portion and the forefoot portion comprise at least one raised section for controlling pronation and/or supination. The at least one raised section preferably has a honeycomb section.

The central portion of each control element described herein preferably extends substantially across the width of the sole when the control element is installed. The central portion of the control element preferably defines a concave region. The concave region is preferably suitable for supporting an arch of a wearer's foot.

The control elements described herein preferably each form a torsion spring. In use, the control element may store energy when it is deformed and subsequently release the energy to provide mechanical assistance. The control element is preferably formed from a resilient material that returns to its original shape.

The control element is preferably moulded from a plastics material. Alternatively, a composite material, such as carbon or glass fibre in combination with a resin, may be used to form the control element. In certain embodiments the control element may be made of or comprise metal.

The forefoot portion of each control element described herein preferably supports the medial and lateral portions of a wearer's foot. The forefoot portion may comprise separate sections for supporting the medial and lateral portions. The forefoot portion may, for example, be bifurcated. Preferably, however, the forefoot portion is substantially continuous and is suitable for supporting both the medial and lateral portions of a wearer's foot.

The control element described herein may form a chassis for the article of footwear.

The present invention also relates to a sole for an article of footwear comprising a control element of the type described herein. A midsole may be provided over the control element to form the sole.

Furthermore, the present invention relates to an article of footwear comprising a control element or a sole as described herein. The article of footwear may be a boot, a shoe, a training shoe or the like.

Unless stated otherwise, references herein to angles are to be understood as being measured in relation to the plane of the ground when the insert is installed in the sole of an article of footwear. Equally, unless stated otherwise, those features of the present invention referred to as being inclined are to be understood as being inclined relative to the plane of the ground when the insert is installed in the sole of an article of footwear.

A preferred embodiment of the present invention will now be described with reference to the accompanying Figures, in which:

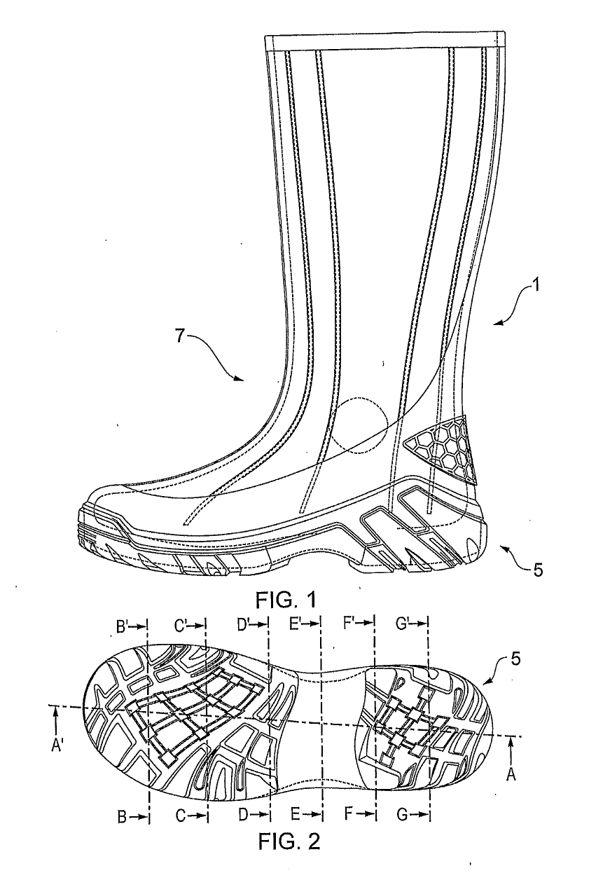

FIG. 1 shows a side view of a boot incorporating a control element in accordance with the present invention;

FIG. 2 shows a plan view of the sole of the boot illustrated in FIG. 1;

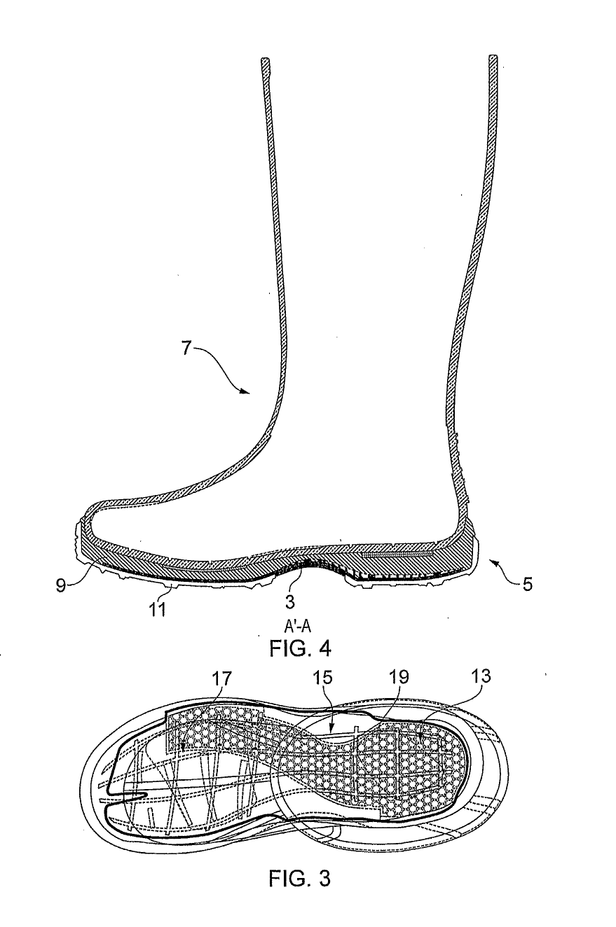

FIG. 3 shows a plan view from above of the boot;

FIG. 4 shows a longitudinal cross-sectional view of the boot along line A-A of FIG. 2; and

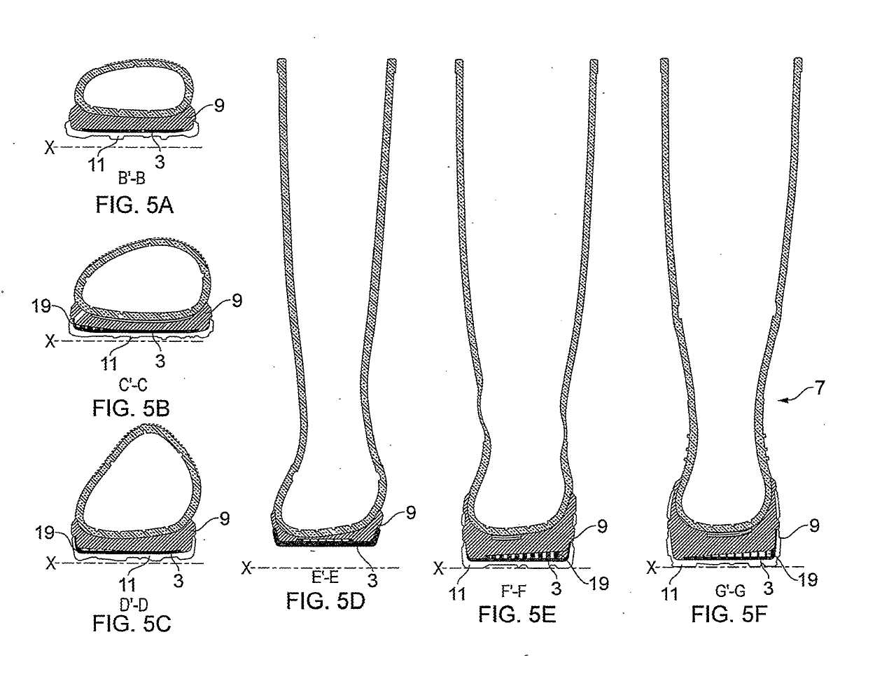

FIGS. 5A-5F show transverse cross-sectional views of the boot along lines B-B, C-C, D-D, E-E, F-F and G-G of FIG. 2.

A waterproof boot 1 in accordance with the present invention is shown in FIGS. 1 and 2. The boot 1 comprises a control element 3, a moulded sole 5 and an upper 7. The control element 3 is moulded from a resilient plastics material and forms a torsion spring capable of storing energy when the sole 5 is deformed. The upper 7 is a one-piece moulding of Ethylene Vinyl Acetate (EVA) formed using injection moulding techniques. The base of the upper 7 forms an insole for the boot 1. It will be appreciated that other plastics materials may be employed to mould the upper 7.

The sole 5 comprises a midsole 9 and an outer sole 11 between which the control element 3 is located. The outer sole 11 provides grip on the base of the boot 1 and is shown in plan form in FIG. 2. The control element 3 in the present embodiment is moulded from a resilient plastics material.

As shown in FIG. 3, the control element 3 comprises a rearfoot portion 13, a central portion 15 and a forefoot portion 17. The rearfoot portion 13 operatively supports the heel of a wearer's foot (not shown). The central portion 15 extends substantially across the width of the sole 5 and is curved to support the arch of the wearer's foot. The forefoot portion 17 is substantially continuous and, in use, provides support for the medial and lateral portions of the wearer's foot.

As shown in FIGS. 3 and 4, a raised section 19 is formed on an upper surface of the control element 3. The effective thickness (or depth) of the raised section 19 varies over the surface of the control element 3 to provide the desired pronation and/or supination control. The greater the effective thickness of the raised section 19 the more support may be provided for the midsole 9 and, hence, a wearer's foot. The raised section 19 has a honeycomb structure, open at its upper end, to reduce the weight of the control element 3.

The raised section 19 is illustrated in FIG. 3 by the dashed lines showing the honeycomb structure. The raised section 19 extends over the entire rearfoot portion 13 of the control element 3, but only along a lateral side of the forefoot portion 17. In use, the raised section 19 in the forefoot portion 17 typically supports the fifth metatarsal bone and optionally also the fourth metatarsal bone. The raised section 19 extends diagonally across the central portion 15 of the control element 3 from a medial side of the rearfoot portion 13 to a lateral side of the forefoot portion 17.

The cross-sectional profile of the control element 3 and the raised section 19 will now be described with reference to FIGS. 5A-F. For ease of reference, angles will be described with reference to a horizontal plane X representative of the plane of the ground.

As shown in FIG. 5A, the raised section 19 does not extend to the front of the control element 3 and the front of the forefoot portion 17 of the control element 3 is substantially planar in the region of the boot 1 defining a toe box. The raised section 19 does however provide an inclined support on the lateral side of the forefoot portion 17 for supporting the lateral metatarsal bones, preferably at least the fourth and fifth metatarsal bones, as shown in FIGS. 5B and 5C. The raised section 19 slopes downwardly in a transverse direction from the lateral side of the forefoot portion 17 towards the medial side at an angle of approximately 2° relative to the horizontal plane X. Thus, the forefoot portion 17 of the control element 3 may help control supination of the wearer's foot. It will be appreciated that the raised section 19 in the forefoot portion 17 may be inclined at an angle in the range 1° to 4°.

The control element 3 is curved in the middle portion 15 to provide support for the arch of a wearer's foot. As shown in FIG. 5D, the control element 3 is substantially parallel to the horizontal plane X over the middle portion 15. Moreover, the raised section 19 provides a substantially horizontal support in the centre of the control element 3.

In the rearfoot portion 13, the raised section 19 slopes downwardly in a transverse direction from the medial side to the lateral side of the rearfoot portion 13 at an angle of approximately 4° relative to the horizontal plane X, as shown in FIGS. 5E and 5F. It will be appreciated that the raised section 19 in the rearfoot portion 13 may be inclined at an angle in the range 4° to 8°.

As outlined above, the control element 3 forms a torsion spring capable of storing energy. Thus, at least in preferred embodiments, the control element 3 serves to control pronation and/or supination of a wearer's foot whilst also assisting the action of walking by releasing stored energy.

For the sake of brevity, the present invention has been described with reference to a boot 1 for a right foot. It will be appreciated that the invention would also typically be embodied in a corresponding boot 1 for a left foot.

Although the present invention has been described with reference to a waterproof boot, it will be appreciated that it may equally applied to other articles of footwear. For example, the control element may be employed in a training or running shoe.

It will be appreciated that various changes and modifications may be made to the present invention without departing from the spirit and scope of the present invention.

1. A control element for installation in a sole of an article of footwear, the control element comprising: a rearfoot portion; a forefoot portion for supporting medial and/or lateral portions of a wearer's foot; and a central portion joining the rearfoot portion and the forefoot portion; wherein said rearfoot portion provides a first inclined support for controlling pronation.

2. A control element as claimed in claim 1, wherein the first inclined support operatively slopes downwardly from a medial side of the control element towards a lateral side thereof.

3. A control element as claimed in claim 1, wherein said rearfoot portion comprises a first raised section that forms said first inclined support.

4. A control element as claimed in claim 1, wherein said first inclined support is operatively inclined at an angle between 2° and 10° relative to the ground.

5. A control element as claimed in claim 1, wherein said first inclined support is operatively inclined at an angle between 4° and 8° relative to the ground.

6. A control element as claimed in claim 1, wherein said forefoot portion forms a second inclined support for controlling supination.

7. A control element as claimed in claim 6, wherein said second inclined support operatively slopes downwardly from a lateral side of the control element towards a medial side thereof.

8. A control element as claimed in claim 6, wherein said forefoot portion comprises a second raised section forming said second inclined support.

9. A control element as claimed in claim 1, wherein said rearfoot portion comprises a first raised section that forms said first inclined support; and said forefoot portion comprises a second raised section forming said second inclined support; wherein said first and second raised sections are joined to form a continuous raised section.

10. A control element as claimed in claim 9, wherein said continuous raised section extends diagonally across the central portion of the control element.

11. A control element as claimed in claim 3, wherein said first raised section and/or said second raised section have a honeycomb structure.

12. A control element for installation in a sole of an article of footwear, the control element comprising: a rearfoot portion; a forefoot portion for supporting medial and/or lateral portions of a wearer's foot; and a central portion joining the rearfoot portion and the forefoot portion; wherein said forefoot portion forms an inclined support for controlling supination.

13. A control element for installation in a sole of an article of footwear, the control element comprising: a rearfoot portion; a forefoot portion for supporting medial and/or lateral portions of a wearer's foot; and a central portion joining the rearfoot portion and the forefoot portion; wherein the rearfoot portion and the forefoot portion comprise at least one raised section for controlling pronation and/or supination.

14. A control element as claimed in claim 13, wherein said at least one raised section has a honeycomb section.

15. A control element as claimed in claim 1, wherein the central portion of the control element extends substantially across the width of the sole when the control element is installed.

16. A control element as claimed in claim 1, wherein the central portion of the control element defines a concave curve.

17. A control element as claimed in claim 1, wherein the control element forms a torsion spring.

18. A control element as claimed in claim 1, wherein the forefoot portion is substantially continuous and is suitable for supporting the medial and lateral portions of a wearer's foot.

19. A sole for an article of footwear comprising a control element as claimed in claim 1.

20. An article of footwear comprising a control element as claimed in claim 1.
